(19) United States
(12) Patent Application Publication
(10) Pub. No.: US 2022/0218925 A1
(43) Pub. Date: Jul. 14, 2022

(54) IMPROVEMENTS RELATING TO INHALERS

(71) Applicant: E-BREATHE LIMITED, Belfast Antrim (GB)

(72) Inventor: David MCLAUGHLIN, Belfast Antrim (GB)

(21) Appl. No.: 17/608,532

(22) PCT Filed: May 7, 2020
(86) PCT No.: PCT/GB2020/051129

§ 371 Date: Nov. 3, 2021

(30) Foreign Application Priority Data
May 9, 2019 (GB) 1906515.0

(51) Int. Cl. A61M 15/00; A61M 11/04; G16H 40/67

(52) U.S. Cl. CPC A61M 15/008; A61M 11/04; G16H 40/67; A61M 2205/8206; A61M 2230/46; A61M 2205/502; A61M 2205/3553; A61M 2205/13

(57) ABSTRACT

The present invention discloses a method of determining the delivery of a dosage from an inhaler able to deliver an active pharmaceutical ingredient (API) as an inhalable aerosol to a user comprising at least the steps of (i) measuring user inhalation airflow during a use occasion of the inhaler to determine the portion of the inhalation above an airspeed threshold F1; (ii) measuring the volume of air inhaled by the user during the use occasion of the inhaler to determine the portion of inhalation occurring prior to a swept volume threshold V1; (iii) determining a first portion P1 of the dosage provided by the inhaler during the use occasion above the threshold F1 and provided prior to threshold V1.

The present invention pertains to a means for delivering inhaled medicaments to a patient, and, in particular, to an apparatus and method of enhancing user compliance with a prescribed dosage regime.

It is well known that the lungs are an efficient entry point for many active pharmaceutical ingredients (APIs) into the body due to their adaptions for gas exchange including large surface area, moist surface, thin outer tissue layer and close proximity to large blood supply and their place within a double circulatory system which reduces the time taken for a API to be distributed to the body vs e.g. intravenous injection.

There are many available means of delivering APIs to the lungs, the most common being dry powder inhalers (DPI) and metered dose inhalers (MDI).

It is well known that deep lung deposition is best for API delivery as the API is deposited within the alveoli where absorption can readily take place. Alveoli deposition is promoted when the API is inhaled in the mid-part of the inhalation cycle rather than supplying a continuous supply of API throughout the inhalation. At the beginning of the inhalation cycle the airflow is ramping up and any API supplied during this part may have insufficient airflow to carry it to the alveoli and hence not be available for absorption. At the end of the inhalation cycle, the API supplied may not be followed by sufficient air volume to be swept from the upper respiratory tract and fully deposited within the alveoli; hence some API may be exhaled rather than absorbed by the patient.

To ensure the maximum amount of the administered dose of API is successfully absorbed by the patient it is therefore desirable to only supply the API during the correct portion of the inhalation cycle. The most desirable portion of the inhalation cycle is when the air flow velocity is sufficient to fully entrain the inhalable aerosol and have sufficient air volume following API administration to fully sweep the API into the aveoli.

In addition, many patients use inhalation apparatus incorrectly. The most common mistakes relate to the inhalation method used and in particular the use of an inappropriate inhalation flow rate (too high or too low), too short an inhalation duration or both. For example, a study by M. Lee-Wong et al. reported that only 5% of patients used an MDI and spacer perfectly, rising to 13% after demonstration. “Results of a programme to improve house staff use of MDI and spacers,” Post Graduate Medical Journal, Vol 79, p 221-225, 2003.

Inhalation apparatus typically come with written instructions to promote correct inhalation technique, however, often these instructions are not read, not understood or not implemented. A patient can be educated in the appropriate inhalation technique; however, this places an additional burden on health care providers. It is beneficial to have a feedback mechanism integrated within an inhalation apparatus to i) provide assurance of correct inhalation technique and ii) indicate inappropriate inhalation technique to promote corrective action.

A further limitation of both MDI and DPI inhalation apparatus is that once activated the complete dose of API is released as a single bolus into the inhalation stream. It is beneficial for an inhalation apparatus to be able to stop API administration if inhalation parameters are not suitable at any point during the inhalation cycle.

Lack of user compliance with a prescribed dosage regime is a significant issue within the healthcare industry. Inhalation apparatus can be fitted with a means of providing a timestamp of each occasion the inhalation device is activated. This timestamp information can be used as a means of providing assurance of user compliance with a prescribing regime. Examples include products by COHERO, PROPELLER and ADHERIUM which act as add-ons to traditional MDI and DPI inhalation apparatus.

A limitation of current compliance information is that should a user activate the inhalation apparatus and not inhale within desirable parameters the API will exit the apparatus yet not be successfully delivered to the deep-lung. This may render the dose of API ineffective yet the compliance information will register a successful administration of API. Hence to enhance the quality of compliance information it is beneficial to provide some record of user inhalation alongside the use occasion of the inhalation device. More beneficial is the provision of information relating to user inhalation during API administration by the device. Most beneficial is the provision of information confirming user inhalation within parameters suitable of deep-lung deposition during the API administration by the apparatus.

It is of further benefit to feedback to a user any use occasions where the inhalation parameters were not desirable and hence the API was likely to be unsuccessfully delivered. Thus encouraging the user to repeat a dose.

A user can receive a partial dose when their inhalation falls outside desirable parameters at some point during the release of the API from the inhaler. It would be beneficial to have a means to establish the portion of the prescribed dose that is successfully administered as this would give a more accurate reflection of the actual dose received by the user compared to counting the number of use occasions the apparatus was activated.

It would be of benefit to establish the portion of the prescribed dose that is unsuccessfully administered during a use occasion and notify the user of any shortfall compared to the prescribed dosage regime. It would be of further benefit to automatically adjust subsequent doses to make up any shortfall compared to the prescribed dosage regime.

It remains evident that there is an unresolved need for an apparatus and method which can remove much of the user error associated with traditional inhalation apparatus and enhance the compliance of the user with prescribed dosage regime by: providing a first pre-determined dosage of API; administering the API when the user inhalation is appropriate; determining any unsuccessfully delivered portion of the first pre-determined dosage; adjusting at least one subsequent pre-determined dose. Optionally the apparatus may notify the user of unsuccessful delivery of at least a portion of the first pre-determined dosage.

One aspect of the present invention provides a method of determining the delivery of a dose or dosage from an inhaler able to deliver an active pharmaceutical ingredient (API) as an inhalable aerosol to a user comprising at least the steps of

(i) measuring user inhalation airflow during a use occasion of the inhaler to determine the portion of the inhalation above an airspeed threshold F1;
(ii) measuring the volume of air inhaled by the user during the use occasion of the inhaler to determine the portion of inhalation occurring prior to a swept volume threshold V1;
(iii) determining a first portion P1 of the dosage provided by the inhaler during the use occasion above the threshold F1 and provided prior to threshold V1;

Optionally, the method further comprises the steps of:

(iv) determining a second portion P2 of the first dosage such that the sum of P1 and P2 equals the first dosage;
(v) determining delivery of the calculated portion P2 of step (iv) during a further delivery of the API from the inhaler.

Optionally step (v) comprises determining to increase one or more subsequent dosages of the API from the inhaler to include the calculated portion P2 of step (iv).

Optionally, step (v) comprises determining to deliver a subsequent dosage of the API wholly or substantially equal to the calculated portion P2 of step (iv).

Optionally, the method further comprises providing feedback of one or more of steps (i)-(v) to the user and/or a remote source.

Optionally, the method further comprises the step of providing feedback to the user to indicate inhalation threshold F1 achieved.

Optionally, the method further comprises the step of providing feedback to the user to indicate swept volume threshold V1 achieved.

Optionally, the method further comprises the step of providing a user with advice on inhalation technique based on the values determined in steps (i)-(v).

According to another aspect of the present invention, there is provided an apparatus capable of determining the delivery of a dosage from an inhaler able to deliver an active pharmaceutical ingredient (API) as an inhalable aerosol to a user, the apparatus comprising: a battery,

an airflow sensor able to measure user inhalation airflow during a use occasion of the inhaler to determine the portion of the inhalation above an airspeed threshold F1 and to measure the volume of air inhaled by the user during the use occasion of the inhaler to determine the portion of inhalation occurring prior to a swept volume threshold V1;
a delivery controlling means able to determine a first portion P1 of the dosage provided by the inhaler during the use occasion above the threshold F1 and provided prior to threshold V1;
a feedback means able to feedback to the user or a remote source;
a communication means; and
an information storage means.

Optionally, the delivery controlling means is also able to determine a second portion P2 of the first dosage such that the sum of P1 and P2 equals the first dosage, and to determine delivery of the calculated portion P2 during a further delivery of the API from the inhaler.

Optionally, the delivery controlling means is able to determine increasing one or more subsequent dosages of the API from the inhaler to include the calculated portion P2.

Optionally, the delivery controlling means is able to determine delivering a subsequent dosage of the API wholly or substantially equal to the calculated portion P2.

Optionally, the apparatus further comprises a vapourisation means and a liquid formulation comprising the active pharmaceutical ingredient (API) in a reservoir.

Optionally, the delivery controlling means is able to release the API only when the airflow sensor measures user inhalation airflow above an airspeed threshold F1.

Optionally, the feedback means includes one or more of the group comprising: visual means, audible means, haptic means, wireless means and electronic means.

The apparatus can be secured to an inhaler able to deliver an active pharmaceutical ingredient (API) as an inhalable aerosol to a user, and can act as a sensing apparatus. In this way, the apparatus is separable from the inhaler which delivers the API.

Optionally, the sensing apparatus is in fluid communication with the air pathway from the inhaler to the user. Optionally, the sensing apparatus is designed to integrate with the inhaler apparatus. Optionally, the sensing apparatus may be shaped to fit over the exit of the inhaler, act as a spacer to elongate the airflow pathway, replace part of the inhaler body or a combination thereof.

In one embodiment prior to a use occasion, the user can combine the sensing apparatus and the inhaler. During a use occasion the sensing apparatus can provide feedback on the characteristics of the user's inhalation airflow, including threshold entrainment airflow F1 and threshold swept volume V1 to provide training on inhalation technique. The sensing apparatus may also provide feedback to the user regarding successfully receiving a prescribed dose or dosage of API during a use occasion or actions to rectify an unsuccessful use occasion or combinations thereof.

In one embodiment, the sensing apparatus would communicate the characteristics of the use occasion to an external system. Data from the sensing apparatus would be reviewed remotely by a clinician or carer and advice provided to the user to promote better compliance with a prescribed dosing regime. Such advice comprises advice on inhalation technique, timing of use occasions and combinations thereof.

In one embodiment the data from the sensing apparatus would be compared to ideal patterns, analysed by computer algorithms, and combinations thereof to establish deviations from the prescribing regime. Such deviations would be notified to the user, a carer, a clinician or combinations thereof.

In alternate embodiment of the present invention the apparatus for sensing airflow is an integral part of the inhaler which delivers the API. This has the added advantage of not requiring any preassembly by the user prior to use. It has a further advantage of integrating the delivery controlling means with the API release from the inhaler. In this embodiment the inhaler can modify subsequent doses of API from the inhaler based on the data from the first use occasion. Thus, any shortfall in API delivered to a user during a first use occasion can be included in a subsequent dose from the inhaler. This shortfall dose can be delivered as a separate dose or combined with at least one other pre-determined dose. This method offers a means to ensure a user receives their prescribed dosage of API within the appropriate time period.

Thus, according to another aspect of the present invention, there is provided an inhaler able to deliver an active pharmaceutical ingredient (API) dosage as an inhalable aerosol to a user, the inhaler comprising a battery, a vapourisation means, a liquid formulation comprising the active pharmaceutical ingredient (API) in a reservoir, an airflow sensor, a delivery controlling means and an information storage means,
wherein the airflow sensor is able to measure user inhalation airflow during a use occasion of the inhaler to determine the portion of the inhalation above an airspeed threshold F1 and to determine the portion of the inhalation prior to a threshold swept volume V1, and
the delivery controlling means is able to determine a first portion P1 of the dosage provided by the inhaler during the use occasion above the threshold F1 and prior to threshold V1.

Optionally, the delivery controlling means is able to determine a second portion P2 of the first dosage of the first dosage such that the sum of P1 and P2 equals the first dosage, and to determine the required delivery of the calculated portion P2 during a further delivery of the API from the inhaler.

Optionally, the delivery controlling means is able to determine an increase in one or more subsequent dosages of the API from the inhaler to include the calculated portion P2 of step (iv).

Optionally, the delivery controlling means is able to determine delivery of a subsequent dosage of the API wholly or substantially equal to the calculated portion P2 of step (iv).

Optionally, the delivery controlling means operates the vapourisation means when the airflow sensor measures user inhalation airflow above an airspeed threshold F1.

Optionally, the inhaler further comprises feedback means, optionally being one or more of visual means, audible means, haptic means, wireless means and electronic means.

According to another aspect of the present invention, there is provided a method of ensuring user compliance with a prescribed dosage regime comprising the use of an apparatus as defined in herein or an inhaler as defined herein.

Optionally, the method further comprises the step of providing feedback to the user to indicate inhalation threshold F1 achieved, and/or to indicate swept volume threshold V1 achieved.

Optionally, the method of user compliance comprises the steps of:

(i) measuring user inhalation airflow during a first use occasion of the inhaler to determine the portion of the inhalation above an airspeed threshold F1;
(ii) measuring the volume of air inhaled by the user during a first use occasion of the inhaler to determine the portion of inhalation occurring prior to a swept volume threshold V1;
(iii) determining the portion P1 of the first dose provided by the inhaler during a first use occasion above the threshold F1 and provided prior to threshold V1;
(iv) determining the portion P2 of the first dose such that the sum of P1 and P2 equals the first dose;
(v) determining delivery of the calculated portion P2 of step (iv) during a further delivery of the API from the inhaler.

Optionally, step (v) comprises increasing one or more subsequent doses of the API from the inhaler to include the calculated portion P2.

Optionally, step (v) comprises providing a subsequent dose of API wholly or substantially equal to the calculated portion P2.

In one embodiment an apparatus and method for delivery of API within an inhalable aerosol is provided that increases at least one subsequent pre-determined dose by up to the amount of dose unsuccessfully delivered during the first use occasion.

In one embodiment an apparatus and method for enhancing user compliance with a prescribed dosage regime is provided wherein the delivery controlling means provides a subsequent pre-determined dose substantially equal to or less than the amount of dose unsuccessfully delivered during the first use occasion.

Successful delivery of API is determined when the user is inhaling in a manner likely to provide deep-lung delivery of the API. The most desirable portion of the inhalation cycle is when the air flow velocity is sufficient to fully entrain the inhalable aerosol and have sufficient air volume following API administration to fully sweep the API into the aveoli. Thus a successful delivery can be defined as occurring when a) API is administered when the user is inhaling with an airflow greater than or equal to a threshold flow rate (F1) and b) when there is sufficient air volume (V1) inhaled after the API is administered to sweep the upper respiratory tract.

An unsuccessful delivery of API is determined when the user fails to inhale in a manner likely to provide deep-lung delivery of the API. Should the user inhalation airflow rate drop below threshold flow rate F1 the API will not be sufficiently entrained within the airflow to travel to the aveoli. Should the API be administered and the inhalation stop prior to the desired sweep volume V1 the API will be deposited within the upper respiratory tract and not be absorbed fully.

When the API is released from the apparatus over a period of time, e.g. 2 seconds, all or part of that dose may be taken successfully. It is of benefit to distinguish between portions of a dose that are successful and those that are unsuccessful.

In one embodiment a method and apparatus is provided that calculates the amount of the pre-determined dose administered successfully P1. In an alternate embodiment a method and apparatus is provided that calculates the amount of pre-determined dose unsuccessful delivered P2. P2 can be calculated by subtracting P1 from the pre-determined dose that was intended to be administered during the use occasion.

In one embodiment, a method and apparatus for delivery of API within an inhalable aerosol is provided that includes at least: a battery, a vapourisation means, a liquid formulation reservoir, an airflow sensor, a control system and an information storage system. Such an apparatus controls the release of the API to a suitable window within the inhalation cycle by i) using one or more airflow sensors to detect inhalation of a suitable rate, ii) energising the vaporisation means to produce an inhalable aerosol from the liquid formulation, iii) stopping the production of inhalable aerosol should the inhalation rate fall outside the suitable range.

In one embodiment, a method and apparatus for delivery of API within an inhalable aerosol is provided that includes at least: i) a vaporisation means to produce an inhalable aerosol from a liquid formulation, ii) an airflow sensor, iii) a delivery controlling means and iv) a feedback mechanism with multiple states. Such method and apparatus can encourage appropriate use of the apparatus by providing the user with feedback using the feedback mechanism. The feedback mechanism provides feedback to the user based on at least one of: (i) the status of the device, (ii) the output of at least one sensor, and (iii) a timing mechanism and combinations thereof.

Feedback provided includes at least one of device ready, desirable inhalation rate achieved, desirable inhalation duration achieved, delivery success, and delivery unsuccessful. Additionally, the feedback mechanism provides feedback on deviations from the desired use protocol including inhalation rate too high or too low and inhalation duration too short.

These and other features of the present invention, as well as the methods of operation and functions of the related elements, will become more apparent upon consideration of the following description and the appended claims with reference to the accompanying drawings, all of which form a part of this specification, wherein like reference numerals designate corresponding parts in various figures. It is to be expressly understood, however, that the drawings are for the purpose of illustration and description only and are not intended as a definition of the limits of the invention.

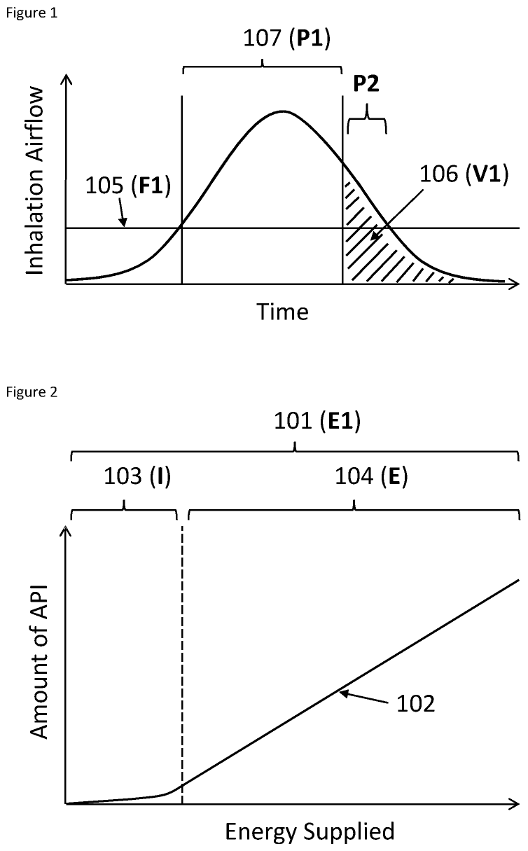

FIG. 1 depicts graphically a typical user inhalation airflow over the course of a use occasion.

FIG. 2 depicts graphically the relationship between energy supplied to the vapourisation means and the amount of API administered by the apparatus.

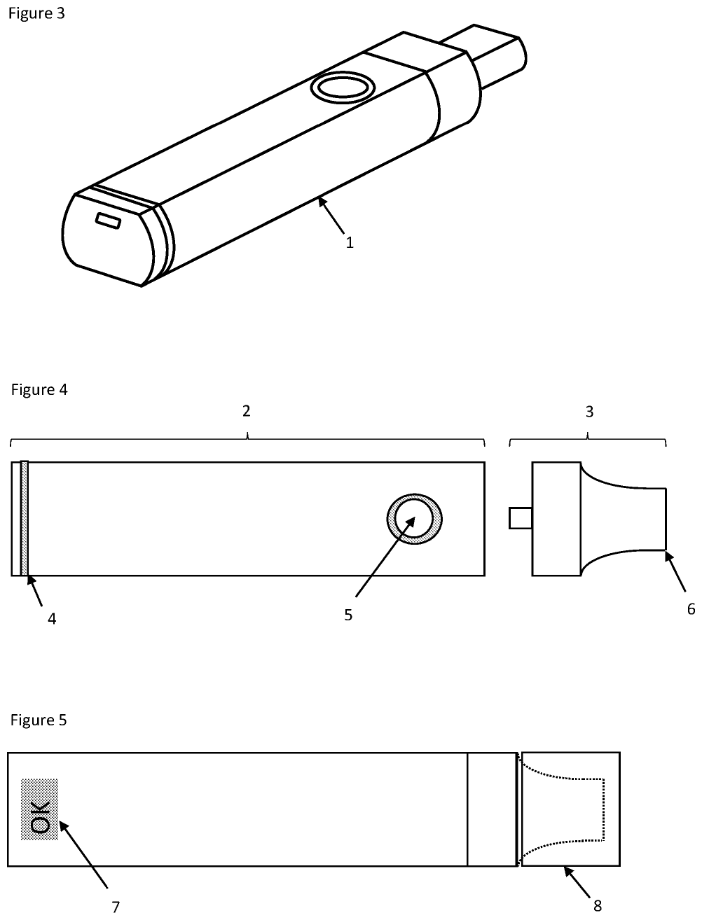

FIG. 3 is an external three-dimensional view of the apparatus according to a particular embodiment.

FIG. 4 is an external plan view of the apparatus according to a particular embodiment.

FIG. 5 is an external plan view of the apparatus according to a particular embodiment.

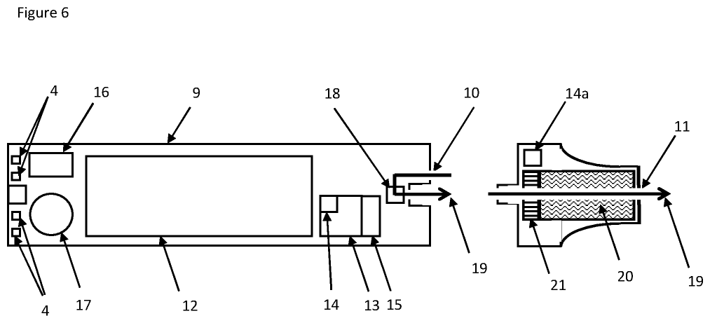

FIG. 6 is a cross-section through the apparatus showing the internal components.

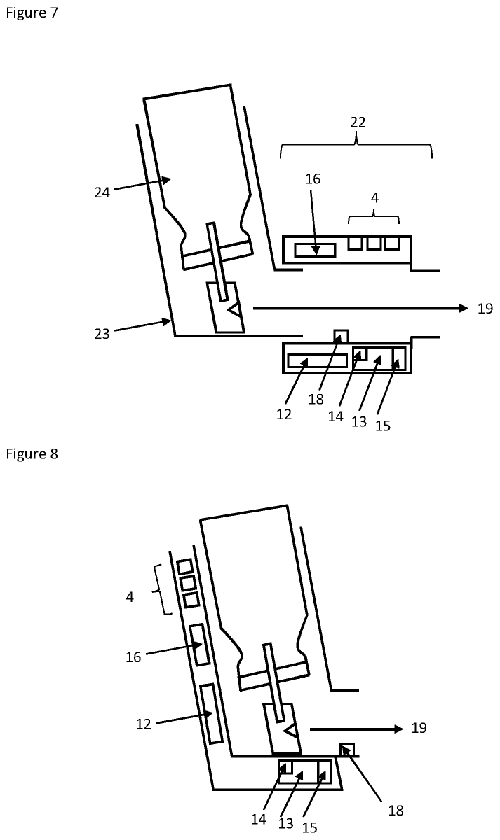

FIGS. 7 and 8 are two cross-sectional views through an apparatus with an inhaler according to further embodiments of the present invention.

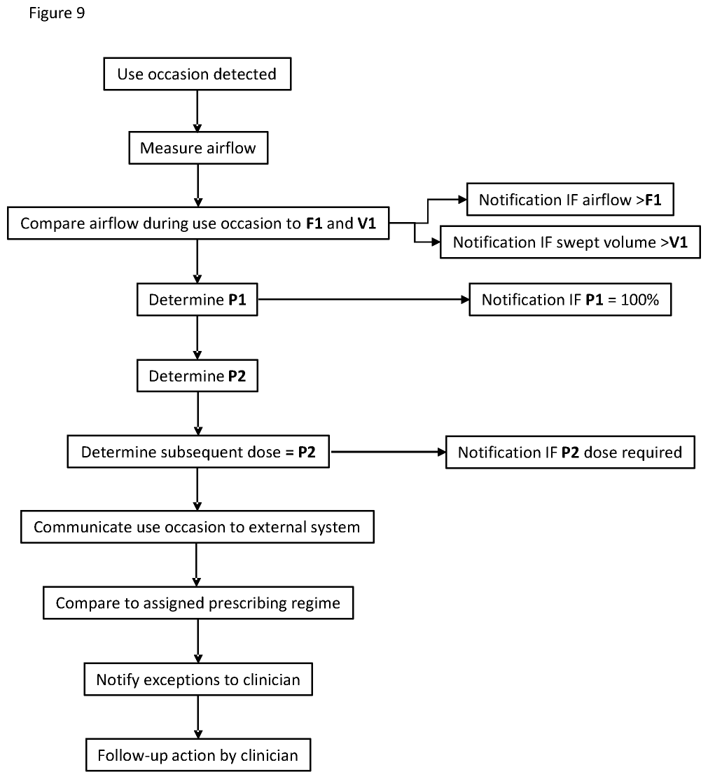

FIG. 9 depicts the operational sequence for a particular embodiment of the apparatus; and

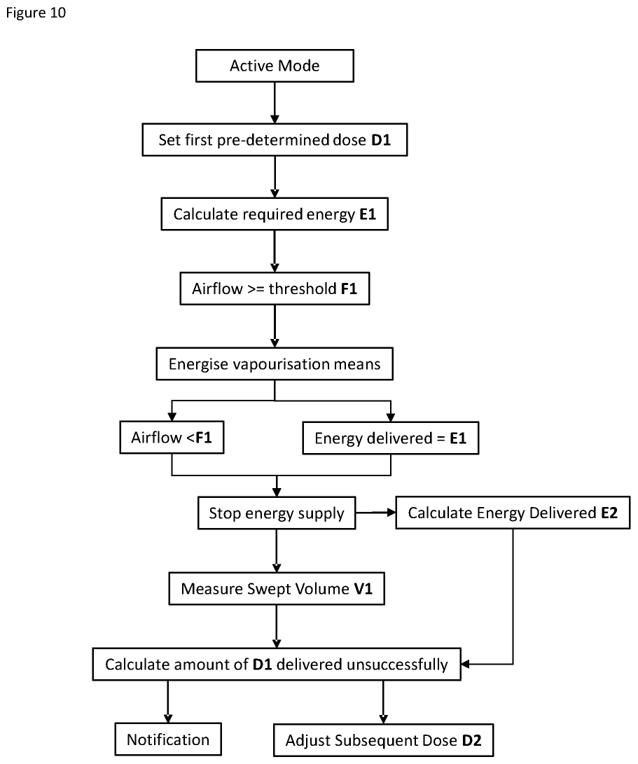

FIG. 10 depicts the operational sequence for an alternative embodiment of the apparatus.

FIG. 1 depicts a typical user inhalation airflow during a use occasion. 105 is the threshold inhalation airflow F1 to sufficiently entrain the inhalable aerosol. 106 indicates the area under the graph that represents the volume of air V1 required to adequately sweep the aerosol into the aveoli. Region 107 represents the portion of the inhalation cycle which is most desirable for effective API delivery to the user.

The portion of the dose P1 administered within region 107 is considered successfully delivered to the user. The portion of the dose P2 administered outside region 107 is considered unsuccessfully delivered to the user.

Threshold inhalation rate F1 is defined as an airflow rate sufficient to entrain the inhalable aerosol produced by the device and carry the aerosol into the lung to be deposited within the alveoli where the active ingredients can be absorbed into the blood stream. Threshold inhalation rate F1 is greater than 0.01 litres per second as measured on a spirometer. Preferably a threshold inhalation rate F1 is greater than 0.05 litres per second. More preferably a threshold inhalation rate F1 is greater than 0.1 litres per second.

Swept volume V1 is defined as a volume of air equivalent to the upper respiratory tract of the user. This volume depends on the user anatomy being greater for those with larger thoracic cavity, e.g. adults typically have greater volume than children. V1 can be in the range 100 to 2000 ml, more preferable V1 is in the range 500 to 1500 ml, more preferably V1 is in the range 750 ml to 1250 ml.

By providing an aerosolization system that sufficiently characterised it is possible to accurately establish the amount of API administered by the apparatus during a use occasion. Of key importance to the accuracy of the estimation of API administration is: i) a known fixed concentration of API within the liquid formulation; ii) a known fixed heat of vapourisation of the liquid formulation; iii) a known thermal mass of the vapourisation means, iv) a known amount of energy supplied to the vapourisation means; v) a known resistance of the vapourisation means. To reduce the complexity of the calculation it is preferable to use a vapourisation means with a constant resistance over operational range of temperature, suitable materials with include NiChrom. To reduce the complexity of the calculation it is preferable to supply energy to the vapourisation means at a constant rate, pulse width modulation is a suitable means to achieve this.

The electrical energy entering the vapourisation means is converted to heat by the heat generative element. This heat has at least three outcomes: i) the temperature of the vapourisation means increases, ii) heat is lost to the surroundings iii) the liquid formulation is volatinised.

FIG. 2 shows how the amount of API administered is related to the amount of energy supplied into the apparatus of the present invention. After a short initial lag period 103 the amount of API is directly proportional to the amount of energy supplied—a steady state system 104. The lag period 103 relates to the energy required to raise the temperature of the vapourisation means and liquid formulation therein to the boiling point of the liquid formulation. Once the vapourisation means is at the boiling point of the liquid formulation essentially all the input energy into the system results in a transition from liquid to vapour under steady state conditions. Once in the vapour phase the API can be considered aerosolised and hence available for delivery.

A sufficiently characterised vapourisation means will have a repeatable lag period 103. The lag period and the energy required to get to a steady state system can both be established experimentally. The energy required to get to a steady state system can also be estimated from the thermal mass of the vapourisation means and liquid formulation contained therein. The energy required is a function of the specific heat capacities of the heated components of the vapourisation means including at least the heat generative means, a portion of the wicking material and a volume of liquid formulation and the temperature change. The temperature change of the vapourisation means during this lag period will be from ambient to the boiling point of the liquid formulation.

To simplify the calculations an estimate of ambient can be made at 20° C. For more accurate calculations a measurement of the starting temperature of the vapourisation means can be made. Temperature measurement can be made directly for example using a thermocouple. Temperature measurement can be made indirectly for example by using a heat generative element whose resistance changes with temperature.

In an embodiment of the present invention, the delivery controlling means establishes the amount of API administered by a calculation using at least: the concentration of API within the liquid formulation, the heat of vapourisation of the liquid formulation and the amount of energy supplied to the vapourisation means.

In an alternate embodiment of the present invention, the delivery controlling means established the amount of API administered by a calculation using at least: the concentration of API within the liquid formulation and the length of time the vapourisation means is energised.

In an embodiment of the present invention, the delivery controlling means establishes the amount of API administered by a calculation using at least: the concentration of API within the liquid formulation, the heat of vapourisation of the liquid formulation and the amount of energy supplied to the vapourisation means less an adjustment for the lag period 103.

In an embodiment of the present invention a method of enhancing user compliance with a prescribed dosage regime is provided that comprises at least the steps of: providing a pre-determined dose of API for a first use occasion; calculating the amount of dose unsuccessfully delivered during the first use occasion; adjusting a least one subsequent dose.

In an embodiment of the present invention a method of enhancing user compliance with a prescribed dosage regime is provided that further comprises increasing a least one subsequent dose by up to the amount of dose unsuccessfully delivered during the first use occasion.

In an alternate embodiment of the present invention a method of enhancing user compliance with a prescribed dosage regime is provided that further comprises providing a subsequent dose of API equal to or less than the amount of unsuccessfully delivered dose from the first use occasion.

FIGS. 3, 4 and 5 show the main external components of the apparatus according to particular embodiments of the present invention. The apparatus 1 is comprised of a main body 2 and a removable liquid formulation reservoir 3. The mouth end 6. FIG. 4 shows the user activated element as a button 5 and the feedback mechanism as an array of LED lights 4. FIG. 5 shows an alternative embodiment where the user activated element is a removable cap 8 and the feedback mechanism is an LCD display 7.

FIG. 6 shows the main internal components of the apparatus: battery 12, delivery controlling means 13, information storage means 14, communication means 15, vibration motor 16 for haptic feedback, microphone 17 for audible feedback and liquid formulation 20. FIG. 6 also shows the outer housing of the device 9 with an air inlet 10 and an air outlet 11 where the user inhales the inhalable aerosol. The pathway for air through the device 19 includes: air inlet 10, airflow sensor 18, vaporisation means 21, air outlet 11.

FIG. 7 shows a cross-section through an embodiment of the sensing apparatus secured to a typical MDI inhaler comprising a plastic shoe 23 and a canister of compressed propellant containing the API as a solute 24. In this embodiment the sensing apparatus 22 is separable from the MDI inhaler.

FIG. 8 shows a cross-section through an alternative embodiment of the sensing apparatus whereby plastic shoe and the sensing apparatus are combined into one integral unit.

FIG. 9 shows a flow diagram of an embodiment of the present invention that would be used by an apparatus shown in FIG. 7 or 8. Upon detection of a use occasion, the airflow sensor measures the user's airflow and compares it to pre-determined threshold values F1 and V1. The apparatus provides feedback to the user via a feedback means of successfully achieving airflow >F1 and swept volume >V1. On completion of the use occasion, the apparatus determines the portion P1 delivered successfully to the user and provides feedback if P1=100% of the pre-determined dose. As required, P2 is determined and feedback given to the user of a requirement for a subsequent dose P2. The apparatus communicates details of the use occasion to an external system. The use occasion is compared to the prescribed dosage regime, any exceptions are notified to the responsible clinician for follow up action such as training on inhaler technique.

The term aerosol shall be interpreted to include gas, vapour, droplets, condensates, particulates and combinations thereof. An inhalable aerosol shall mean an aerosol with an average particle size as measured by laser dispersion ranging from 0.1 to 10 μm, more preferably 0.1 to 1.5 μm.

Liquid formulation 20 shall be interpreted to include liquids, mixtures, solutions, suspensions, micelles, gels, foams, mousses and combinations thereof. Additionally, the liquid formulation can be contained within a matrix, absorbed within a matrix or adsorbed onto a matrix and combinations thereof. Suitable matrices include absorbent fabrics such as cotton or glass wool and solid adsorbents such as zeolites and other inorganic clays.

Battery 12 shall be interpreted as any means of storing an electrical charge including metal-acid accumulators, cells based on zinc, nickel or lithium wherein the electrolyte is liquid, solid or polymeric in nature. Alternatively, a capacitor can also be used as a means of storing electrical charge. Of particular relevance to the present invention are lithium-polymer rechargeable batteries such as those based on lithium iron phosphate and lithium manganese oxide.

A vaporisation means 21 shall be interpreted to be any means of converting the liquid formulation 20 into an aerosol. In a preferred embodiment, the vaporisation means 21 utilises a heat generative element to generate heat energy which converts the liquid formulation into a vapour. This vapour subsequently condenses to form droplets which are suitable for inhalation. The heat generative element converts electrical energy derived from the battery 12 into heat. Heat is produced as a result of the resistive nature of the heat generative element. The heat generative element can be composed of a resistive metal such as titanium and stainless steel or a metal alloy and combinations thereof. Preferably the heat generative element contains the alloy NiChrom which is desirable as it has a constant resistance at a range of temperatures. Alternatively, the heat generative element can be composed of a resistive ceramic such as those based on alumina or silicon nitride.

A vaporisation means 21 is further characterised by being in fluid connection with the liquid formulation 20 to provide a supply of liquid for vaporisation. The connection between the vaporisation means 21 and the liquid formulation 20 is by a wicking means such as a wick, capillary system or tube capable of transferring liquid. Of particular relevance to the present invention are materials that interact with the liquid formulation by capillary action. Such materials act both to transfer liquid to the heat generative means by forming a continuous liquid path and act as a barrier to prevent undesirable liquid leakage from the device due to their ability to retain liquid within their structure.

An airflow sensor 18 is any system capable of detecting the movement of air through the device and providing an electrical communication to the delivery controlling means 13. Airflow sensor 18 can be interpreted to mean a single sensor or multiple sensors. In an embodiment of the present invention one sensor is used to detect an air flow rate and a second sensor detect a higher air flow rate, the combination of both sensor outputs is then used to determine air flow within a desirable range. Additional air flow ranges can be determined by the appropriate use of different sensing levels with one or more sensors. A preferred embodiment utilises a single sensor with multiple sensing thresholds that can provide electrical communication corresponding to the different air flows. An airflow sensor can measure airflow using a rotating vane anemometer, a moving vane meter, a hot-wire detector, a Kármán vortex sensor, an electromechanical membrane sensor, MEMS technology or combinations thereof.

A preferred embodiment of the present invention utilises an airflow sensor 18 containing a capacitive microphone to detect air flow. The flow of air through a device alters air pressure and generates turbulence which deflects a charged diaphragm within a microphone causing a change in capacitance. The change in capacitance is detected electronically and used to generate a communications signal to the delivery controlling means.

An alternative preferred embodiment of the present invention utilises a MEMS pressure sensor as an airflow sensor 18. The action of the user inhaling through the apparatus causes a reduction in air pressure which is converted into an electrical signal by the MEMS sensor and the signal is passed to the delivery controlling means. Higher flow rates cause a greater reduction in air pressure, hence within a defined airflow pathway such MEMS sensor can be accurately calibrated to measure user inhalation air flow.

A user activated element 5, 8 is a means by which a user can interact with the device to bring a change from sleep mode to active mode. Preferably a user activated element is a means to alter an electrical circuit such which communicates with the delivery controlling means to activate the device. A user activated means may be a button, switch, lever, contacts, touch switch reliant upon capacitance, resistance or piezo or combination thereof. Preferably a user activated element is a depressible button 5. It is advantageous that the design of the user activated element prevents accidental activation or activation by a minor. Such accidental activation can be prevented by using mechanically complexity or more preferably by requiring a particular sequence of button presses such as five presses within two seconds to cause activation.

In an alternative preferred embodiment, a user activated element is a physical barrier which prevents use of the device unless moved. The action of moving the physical barrier from its resting position is preferably linked to the actuation of an electrical means which communicates with the delivery controlling means. The physical barrier can be separable from the device or be conjoined via a joining element. A separable user activated element can be a removable case, housing or sleeve. In an alternative embodiment, the physical barrier is mechanically complex which is useful to prevent unintended usage of the device by minors such as a cap 8 which can be child resistant.

A delivery controlling means 13 shall be interpreted as electronic circuitry which can respond to communication signals from the airflow sensor 18, activate the vaporisation means 21 and alter the state of the feedback mechanism 4, 7. A delivery controlling means can perform various calculations including but not limited to calculating P1 and P2. Additionally, a delivery controlling means can also respond to a communication signal from the user activated element 5, 8. Additionally, a delivery controlling means can also activate the airflow sensor 18. A delivery controlling means 13 typically utilises at least one microprocessor to process the communications, perform calculations, actuate elements and alter the feedback mechanism.

In an embodiment of the present invention the delivery controlling means 13 interacts with a removable liquid formulation reservoir 3 and thereby modifies at least one vaporisation parameter including temperature, time, duration and combinations thereof, the relevant parameters being stored within an information storage means 14a within the removable liquid formulation reservoir, in a library referenced by the delivery controlling means or combinations thereof.

A delivery controlling means 13 can be in communication with an information storage means 14, 14a. Preferably an information storage means is a solid-state memory. The information storage means can be part of the main apparatus body 14. The information storage means can be part of the liquid formulation reservoir 14a. Preferably both the main apparatus body and the liquid formulation reservoir contain information storage means.

Preferably the delivery controlling means can communicate externally to provide electronic feedback via a plug-in wired interface using a standard protocol such as USB. Preferably the delivery controlling means can communicate externally using a communication means 15 using means such as Bluetooth, WiFi, LoRA, radiowave, microwave, infra-red and combinations thereof to provide wireless feedback. Preferably external communications are two-way providing data to the external system and receiving data from the external system.

Data to be provided by the delivery controlling means to the external system includes use events and device information. A use event means any interaction between the user and the device relevant to the purpose of the invention and any resultant event caused by that action. A use event includes removal of a cap, insertion of a liquid formulation reservoir, actuation of a user activated element, inhalation, achievement of the suitable inhalation flow rate, achievement of the desired duration of inhalation, activation of the vaporisation means, status of feedback mechanism, successful delivery of API, unsuccessful delivery of API, amount of pre-determined dose successfully delivered P1; amount of pre-determined dose unsuccessfully delivered P2 and combinations thereof.

Device information includes identifiers and version numbers of device hardware, firmware, software; identifiers for removable liquid formulation reservoir; amount of battery capacity and liquid formulation used and remaining, fault codes, system status, system time and combinations thereof.

Data to be received by the delivery controlling means from the external system includes prescription information, prescribed dosage regimes, software updates, firmware updates, fault diagnosis, fault resetting, system resetting, information regarding the liquid formulation and the liquid formulation reservoir, parameters for vaporisation and combinations thereof.

In an embodiment of the present invention the delivery controlling means receives instructions from an external system and thereby modifies at least one vaporisation parameter including temperature, time, duration, delay and combinations thereof.

A communication means can communicate the amount of API successfully delivered during a use occasion to an external system. A communication means can communicate the amount of API unsuccessfully delivered during a use occasion to an external system.

A feedback mechanism is any means for the device to communicate with the user to confirm or indicate device status including visual, auditory, haptic means and combinations thereof. A feedback mechanism has at least two states that the delivery controlling means switches between. More preferably a feedback mechanism has multiple states that can be activated by the delivery controlling means. Preferably the feedback means comprises at least two of visual means, audible means and haptic means.

A feedback means can provide feedback to the patient to indicate suitable inhalation rate achieved and suitable inhalation duration achieved using a feedback mechanism. A feedback means can provide feedback on the amount of API successfully delivered during a use occasion. A feedback means can provide feedback on the residual amount of API unsuccessfully delivered during a use occasion.

An embodiment of the present invention comprises an apparatus and method for enhancing user compliance with a prescribed dosage regime comprises at least a battery, a vapourisation means, a liquid formulation reservoir, an airflow sensor, a delivery controlling means, an information storage means, a feedback means and a communication means.

A preferred embodiment of the present invention uses at least one light emitting diode 4 (LED) to provide visual feedback. The multiple states for visual feedback include turning on, turning off, change in intensity, change in colour of the at least one LED and combinations thereof. In a preferred embodiment, more than one LED is used to provide visual feedback. An alternative embodiment uses at least one liquid crystal display 7 (LCD) to provide visual feedback, more preferably an array of LCD such as a seven segment LCD which can be used to display alpha numeric characters. Alternate display technologies such as those found in consumer electronic apparatus can also be used to provide visual feedback.

An alternate preferred embodiment of the present invention uses at least one speaker 17 to produce audible feedback. The multiple states for audible feedback include turning on, turning off, change in intensity, change in pitch of sound emitted, verbal messages, and combinations thereof.

An alternate preferred embodiment of the present invention uses at least one vibration motor 16 to produce haptic feedback. The multiple state for haptic feedback include turning on, turning off, change in intensity, change in pitch of vibrations emitted and combinations thereof.

More preferably the feedback mechanism uses visual feedback and at least one other feedback means such as audible or haptic or both. This is useful for visually impaired users.

The term active pharmaceutical ingredient (API) shall be interpreted as any chemical which has a pharmacological or sensorial effect. The terms drug and medicament are hereby included within this definition of API.

Optionally the API may comprise tobacco, extracts of tobacco (by water or organic solvent), nicotine, taurine, clove and combinations thereof.

Optionally the API may comprise: cetirizine, pseudoephedrine, ibuprofen, naproxen, omeprazole, doxylamine, diphenhydramine, melatonin, or meclizine and combinations thereof.

Optionally the API may comprise: albuterol, levalbuterol, pirbuterol, salmeterol, formoterol, atropine sulfate, ipratropium bromide, fluticasone, budesonide, mometasone, montelukast, zafirlukast, theophylline or combinations thereof.

Optionally the API may comprise: a polyphenol, a green tea catechin, caffeine, a phenol, a glycoside, a labdane diterpenoid, yohimbine, a proanthocyanidin, terpene glycoside, an omega fatty acid, echinacoside, an alkaloid, isovaleric acid, a terpene, gamma-aminobutyric acid, a senna glycoside, cinnamaldehyde, Vitamin D or combinations thereof.

Optionally the API may comprise organic material from a Cannabis genus plant, an extract from a Cannabis genus plant, a cannabinoid or combinations thereof. The API may comprise tetrahydrocannabinol (THC), carmabigerolic acid, cannabigerol, tetrahydrocannabinolic acid, cannabichromene, cannabicyclol, cannabivarin, cannabichromevarin, cannabigerovarin, cannabigerol monomethyl ether, delta-8-tetrahydrocannabinol, delta-9-tetrahydrocannabinol, tetrahydrocannabivarin, cannabinolic acid, cannabinol, cannabidiolic acid, cannabidivaric acid, cannabidiol (CBD), cannabichromenic acid, cannabichromene, cannabicyclolic acid or combinations thereof.

In a preferred embodiment the API is CBD. In an alternative embodiment the API is THC. In an alternative embodiment the API is a combination of THC and CBD.

In a preferred embodiment of the present invention the outer body of the device 9 is made of acrylonitrile butadiene styrene plastic; the airflow sensor 18 is a Pressure sensor by ST Micro; the battery 12 is a lithium polymer cell 3.7 v 840 mAh by YOK; the feedback mechanism 4 is an array of four LEDs and a vibration motor 16; the liquid formulation 20 is a 1 millilitre solution of 200 mg per millilitre CBD in a 80:20 mix of propylene glycol and glycerine; the vaporisation means 21 comprises a heat generative element composed of Nichrome wire of resistance 2 ohm, wrapped helically around a central glass fibre wick separated from the liquid formulation 20 by a pad of cotton wicking material; the body of the liquid formulation reservoir is made of polyethylene terephthalate; the user activated element 5 is a push-to-make depressible button; the delivery controlling means 13, communication means 15 and information storage means 14 are an integrated unit based on a Nordic Semiconductor Bluetooth SOC and an Atmel 8 bit AVR Microcontroller, with the circuit completed using appropriate components and coded appropriately by those skilled in the art.

In this preferred embodiment, the desirable inhalation threshold F1 is set at 0.025 litres per second and the desirable swept volume V1 is set at 250 ml.

Following the flow diagram depicted in FIG. 10, in this preferred embodiment, the user activates the apparatus 1 using button 5.

The delivery controlling means 13 calculates the total amount of energy required E1 to deliver the first dose of API D1. This utilises a stored value for the initial energy (I) related to the lag period 103 and the actual energy required (E) during the steady state 104 calculated from the specific heat of vapourisation of the liquid formulation 20, the concentration of API within said formulation and the dose D1 of API required.

Upon sensing user inhalation airflow greater than desired threshold F1 the vapourising means 21 is energised and an inhalable aerosol is produced from the liquid formulation 20. Provided the inhalation airflow remains greater than F1, the delivery controlling means continues to supply energy to the vapourisation means until the total amount of energy supplied is equal to the total energy E1 calculated.

Should the airflow fall below F1 the supply of energy is discontinued. After the supply of energy is finished, the airflow sensor 18 continues to measure user inhalation airflow until it ceases.

The delivery controlling means sums the energy supplied E2 to the vapourisation means during the period when inhalation flow rate >F1 and swept volume >V1.

If E2=E1 then all API is recorded as successfully delivered and P1 equals 100%. If E2<E1, the delivery controlling unit establishes the difference between E1 and E2 and calculates the portion of API unsuccessfully delivered P2.

As required, the delivery controlling means notifies the user of an unsuccessful portion of API administered P2 and the need for an additional dose using the feedback mechanism 4.

The unsuccessful portion P2 of the first dose calculated is then used as the subsequent dose D2 and the process repeated.

In this preferred embodiment, the delivery controlling means 13 captures data relating to the date, time and characteristics of each use event, device and liquid formulation reservoir identities, stores it using the information storage means 14 makes the data available via the communication means 15 to an external system via Bluetooth once a connection becomes available. In this embodiment the delivery controlling means also stores use event data on the information storage means 14a within the liquid formulation reservoir 3.

Although the invention has been described in detail for the purpose of illustration based on what is considered to be the most practical and preferred embodiment, it is to be understood that such detail is solely for that purpose and that the invention is not limited to the disclosed embodiments, but, on the contrary, it is intended to cover modifications and equivalent arrangements that are within the scope of the appended claims. For example, it is to be understood that the present invention contemplates that, to the extent possible, one or more features of any embodiment can be combined with one or more features of any other embodiment.

1. A method of determining the delivery of a dosage from an inhaler able to deliver an active pharmaceutical ingredient (API) as an inhalable aerosol to a user comprising at least the steps of
(i) measuring user inhalation airflow during a use occasion of the inhaler to determine the portion of the inhalation above an airspeed threshold;
(ii) measuring the volume of air inhaled by the user during the use occasion of the inhaler to determine the portion of inhalation occurring prior to a swept volume threshold;
(iii) determining a first portion of the dosage provided by the inhaler during the use occasion above the threshold and provided prior to threshold.

2. The method according to claim 1 further comprising the steps of
(iv) determining a second portion of the first dosage such that the sum of P1 and P2 equals the first dosage;
(v) determining delivery of the calculated portion of step (iv) during a further delivery of the API from the inhaler.

3. The method according to claim 2 wherein step (v) comprises determining to increase one or more subsequent dosages of the API from the inhaler to include the calculated portion of step (iv).

4. The method according to claim 2 wherein step (v) comprises determining to deliver a subsequent dosage of the API wholly or substantially equal to the calculated portion of step (iv).

5. The method according to claim 1, further comprising providing feedback of one or more of steps (i)-(v) to the user.

6. The method according to claim 1, further comprising communicating one or more of steps (i)-(v) to a remote source.

7. The method according to claim 1, further comprising the step of providing feedback to the user to indicate inhalation threshold achieved.

8. The method according to claim 1, further comprising the step of providing feedback to the user to indicate swept volume threshold achieved.

9. The method according to claim 1, further comprising the step of providing a user with advice on inhalation technique based on the values determined in steps (i)-(v).

10. An apparatus capable of determining the delivery of a dosage from an inhaler able to deliver an active pharmaceutical ingredient (API) as an inhalable aerosol to a user, the apparatus comprising:
a battery,
an airflow sensor able to measure user inhalation airflow during a use occasion of the inhaler to determine the portion of the inhalation above an airspeed threshold and to measure the volume of air inhaled by the user during the use occasion of the inhaler to determine the portion of inhalation occurring prior to a swept volume threshold;
a delivery controlling means able to determine a first portion of the dosage provided by the inhaler during the use occasion above the threshold and provided prior to threshold;
a feedback means able to feedback to the user or a remote source;
a communication means; and
an information storage means.

11. The apparatus according to claim 10 wherein the delivery controlling means is also able to determine a second portion of the first dosage such that the sum of the first portion and the second portion equals the first dosage, and to determine delivery of the second portion during a further delivery of the API from the inhaler.

12. The apparatus according to claim 11 wherein the delivery controlling means is able to determine increasing one or more subsequent dosages of the API from the inhaler to include the second portion.

13. The apparatus according to claim 11 wherein the delivery controlling means is able to determine delivering a subsequent dosage of the API wholly or substantially equal to the second portion.

14. The apparatus according to claim 10, further comprising a vapourisation means and a liquid formulation comprising the active pharmaceutical ingredient (API) in a reservoir.

15. The apparatus according to claim 10, wherein the delivery controlling means is able to release the API only when the airflow sensor measures user inhalation airflow above an airspeed threshold.

16. The apparatus according to claim 10, wherein the feedback means includes one or more of the group comprising: visual means, audible means, haptic means, wireless means and electronic means.

17. The apparatus according to claim 10 secured to an inhaler able to deliver an active pharmaceutical ingredient (API) as an inhalable aerosol to a user.

18. An inhaler able to deliver an active pharmaceutical ingredient (API) dosage as an inhalable aerosol to a user, the inhaler comprising a battery, a vapourisation means, a liquid formulation comprising the active pharmaceutical ingredient (API) in a reservoir, an airflow sensor, a delivery controlling means and an information storage means,
wherein the airflow sensor is able to measure user inhalation airflow during a use occasion of the inhaler to determine the portion of the inhalation above an airspeed threshold and to determine the portion of the inhalation prior to a threshold swept volume, and
the delivery controlling means is able to determine a first portion of the dosage provided by the inhaler during the use occasion above the threshold and prior to threshold.

19. The inhaler as claimed in claim 18 wherein the delivery controlling means is able to determine a second portion of the first dosage of the first dosage such that the sum of the first portion and the second portion equals the first dosage, and to determine the required delivery of the second portion during a further delivery of the API from the inhaler.

20. The inhaler as claimed in claim 18 wherein the delivery controller means is able to determine an increase in one or more subsequent dosages of the API from the inhaler to include the second portion.

21. The inhaler as claimed in claim 18 wherein the delivery controller means is able to determine delivery of a subsequent dosage of the API wholly or substantially equal to the second portion.

22. The inhaler according to claim 18, wherein the delivery controller means operates the vapourisation means when the airflow sensor measures user inhalation airflow above an airspeed threshold.

23. The inhaler according to claim 18, further comprising feedback means, optionally being one or more of visual means, audible means, haptic means, wireless means and electronic means.

24. (canceled)
